(19) United States
(12) Patent Application Publication
(10) Pub. No.: US 2005/0064427 A1
(43) Pub. Date: Mar. 24, 2005

(54) Method and/or system for identifying fluorescent, luminescent and/or absorbing substances on and/or in sample carriers

(76) Inventors: Martin Gluch, Jena (DE); Ralf Wolleschensky, Apola (DE)

Correspondence Address: Gerald H Kiel;Reed Smith, 599 Lexington Avenue, New York, NY, 10022-7650, US

(21) Appl. No.: 10/500,648

(22) PCT Filed: Dec. 14, 2002
(86) PCT No.: PCT/EP02/14275

(30) Foreign Application Priority Data
Jan. 3, 2002 (DE) 102 00 499.4

(51) Int. Cl. C12Q 1/68; G01N 33/558

(52) U.S. Cl. 435/6; 436/514

(57) ABSTRACT

Method and/or arrangement for identifying fluorescing, luminescing and/or absorbing substances on and/or in sample carriers, particularly with high sample throughput in sample screening and/or in diagnostics, preferably in the analysis of samples in microtiter plates (MTP), wherein a spectral splitting of the sample light is carried out and detection is carried out in a plurality of detection channels, and at least one summation and/or combination of the signals of the individual channels is carried out for at least a portion of the detection channels. In an advantageous manner, at least one standard sample (STD) and/or at least one blank sample (BLK) are/is arranged on the sample carrier in addition to the substances (PRB) to be examined, and a spectrum of at least one standard sample (STD) is recorded in a first step.

### CROSS-REFERENCE TO RELATED APPLICATIONS

This application claims priority of International Application No. PCT/EP02/14275, filed Dec. 14, 12002 and German Application No. 102 00 499.4, filed Jan. 3, 2002, the complete disclosures of which are hereby incorporated by reference.

### 1. BACKGROUND OF THE INVENTION

a) Field of the Invention

The invention is directed to a method and an arrangement for the identification of chemically active substances on or in sample carriers. The simultaneous reception of complete spectral bands opens up novel possibilities for discriminating between active chemical substances and false positive interference signals. This is highly significant particularly in applications requiring a high sample throughput (high throughput screening, diagnostics).

b) Description of the Related Art

1.1 Microplates

In chemical analysis, the microtiter plate has become the established standard in sample carriers for the analysis of samples. Microtiter plates with 96, 384 or 1,536 cavities (wells) make it possible to prepare and analyze a corresponding quantity of samples simultaneously. The format of these sample carriers is prescribed by the SBS standard (www.sbs.org).

1.2 Assays

Another important aspect apart from the handling of samples (addition of solutions, mixing, incubation) is the analysis of chemical reactions. There are many different optical parameters used for this application as measured quantities. In pharmaceutical active ingredient research in particular there are many different analysis procedures (assays). Examples of physical measured quantities include fluorescence intensity (FL), fluorescence polarization (FP), fluorescence energy transfer (FRET), fluorescence lifetime (FLD), luminescence (LUM), and absorption (ABS). A survey of different assay formats may be found in “High Throughput Screening: the discovery of bioactive substances”, John P. Delvin, ed., Marcel Dekker, Inc., 1997, Chapter 15. Examples of specific assays are described in Chapters 17, 18, 19, 22, 23 and 24.

1.3 Simultaneous Measurement of a Plurality of Wavelengths

In simple assays, measurement and evaluation are carried out in one wavelength. However, there are also formats in which several wavelengths are necessary for analysis. Following are examples of such formats:

1. Ratio Imaging. Reversible interaction with the specific bonding partners (ligands such as Ca2+, Mg2+, H+) results in a spectral shift of the fluorescence emission of these dyes (Indo-1, SARF; Molecular Probes, Inc., see FIG. 11b). In general, two states of the dye contribute to the emission spectrum that is measured at a given time in the observed volume: FF (free dye) and FB (dye with bonded ligands) (FIG. 11a), whose relative proportions in a given volume are characterized in turn by the affinity between dye and ligand described by the dissociation constant (KD). Therefore, the ratio of the fluorescence of FF and FB represents a measure of the concentration of ligands.

2. FRET. FRET (fluorescence resonance energy transfer) is the radiationless transfer of photon energy from an excited fluorophore (donor) to another fluorophore (acceptor). Overlapping of the donor emission and acceptor emission spectra and close spatial association of donor and acceptor are among the preconditions. In biomedical applications, the FRET effect is used, for example, to determine and track concentrations of ions or metabolites (Ca2+, cAMP, cGMP) or other, e.g., ligand-dependent, structural changes (e.g., phosphorylation state of proteins, conformational changes of DNA). This is achieved by coupling the FRET partners, donor and acceptor (e.g., the synthetic fluorochromes FITC and rhodamine or the genetically coded, fluorescing proteins CFP and YFP) to a molecule undergoing changes in its secondary structure due to specific interaction with the ions, metabolites or ligands under observation (e.g., Miyawaki, et al., Proc Natl Acad Sci USA 96, 2135-2140, March 1999, see FIG. 13) or to one of two molecules which interact permanently or depending on environmental conditions. FIG. 12 shows the emission spectrum for the FRET partners for different distances between donor and acceptor (a—large distance, no FRET/b—short distance, FRET interaction). In both cases, differences in the proportions of the free and ligand-bonded FRET systems in the observed volume are connected with spectral differences in the fluorescence emission of the FRET system. With preferred excitation of donor fluorescence, increases or decreases in ligand bonding are expressed in opposite changes in the amplitudes of the fluorescence emissions of the two FRET partners.

3. Homogeneous Time Resolved Fluorescence. The ratio of two wavelengths (in the present instance, 620 nm and 665 nm) is also used in this case to measure the signal. A description is found in “High Throughput Screening: the discovery of bioactive substances”, John P. Delvin, ed., Marcel Dekker, Inc., 1997, Chapter 19.

1.4 High Throughput Screening

The development of a new medicine in pharmaceutical active ingredient research begins with the search for a chemically active substance which interacts with a specific biological molecule. This substance is then used for further optimization until the medicine is achieved. The interaction is detected by means of an assay through which the reaction can be detected by a physically measurable signal. In primary screening, as it is called, the activity of different substances (up to several hundred thousand) is investigated in the assay. The aim is to select the substances with the greatest activity. The activity is not measured as an absolute value but rather in relation to the activity of a known substance referred to as a standard or 100% control. A similar procedure is also used in diagnostics; instead of substances from substance libraries, patient samples are examined in an assay for determined disease parameters.

1.5 Quality of Measurements

It is essential for the processes described above that the measurement always relates to a control substance. In order to avoid erroneous results due to changes in the way the process is conducted, these controls are also measured in each assay, e.g., by adding these controls to some of the wells in the microplate. The measured signal is converted to a relative signal strength in the following manner:
Relative signal substance=(measured signal substance−measured substrate)/(measured signal standard−measured substrate). The substrate is a measurement of the signal in the solution when no substance or control is added (blank measurement). This blank measurement is carried out in the same way as for the standard in that they are determined for every assay, e.g., through the use of corresponding wells on the microplate for this purpose.

In practice, it is desirable to achieve the greatest possible usable measurement range between the substrate signal (which defines the detection limit) and the signal of the control. An essential feature for the quality of an assay is the specificity of the detection reaction. The more specific the assay, the fewer false positive or false negative results will be obtained. A false positive signal is when the measured signal is positive but no reaction has taken place. This can be the case, for example, in an FL assay, when the investigated substance has autofluorescence or, in an ABS assay, when the substance is colored. A false negative signal is when no signal has been measured but a reaction has taken place. This can also be caused by physiochemical characteristics of the individual samples.

1.6 Microplate Reader

Microplate readers are used for detecting the physical measurements of the assay. These devices are comparable in construction to photometers or fluorometers which illuminate a well by transmitted light or incident light and record the measurement value by means of a light-sensitive sensor. The selection of the specific wavelength range for the investigation is made by means of color filters or interference filters. In order to detect a plurality of wavelengths simultaneously, these wavelengths are measured sequentially. The associated wavelength is realized by means of a selectable bandpass filter. Alternatively, a plurality of detectors are used and the corresponding wavelength range is selected, per detector, by a splitter mirror (see “High Throughput Screening: the discovery of bioactive substances”, John P. Delvin, ed., Marcel Dekker, Inc., 1997, page 357). The disadvantage in the methods mentioned above consists in that the number of selectable detection bands and their bandwidth is predetermined by the filter being used and measurement of more than two wavelength bands is time-consuming. A short measurement time is advantageous in particular when measuring reaction kinetics. With different dyes having overlapping wavelength regions, the bandwidth of the detection filter must be selected so as to be as small as possible according to the prior art in order to minimize crosstalk of adjacent signals as far as possible. As a result, the intensity of the detected signal is very small due to the small bandwidth.

For free selection of excitation and emission ranges, there are also readers which are provided with a grating monochromator which enables a free selection of the wavelength of the excitation or emission light depending on the arrangement. With these arrangements, a spectrum of the detected light can be recorded for each well. However, this process is also sequential because of the scanning process of the monochromator.

### SUMMARY OF THE METHOD AND ARRANGEMENT ACCORDING TO THE INVENTION

In accordance with the present invention, a method and/or arrangement for identifying fluorescing, luminescing and/or absorbing substances on and/or in sample carriers, particularly with high sample throughput in sample screening and/or in diagnostics, preferably in the analysis of samples in microtiter plates wherein a spectral splitting of sample light is carried out and detection is carried out in a plurality of detection channels and at least one summation and/or combination of the signals of the individual channels is carried out for at least a portion of the detection channels.

1.7 Advantages of the Method and Arrangement according to the Invention

The method according to the invention has the following advantages over previous methods: The quantity of dye signatures that may be used simultaneously, i.e., the quantity of characteristics, for example, of cells that can be investigated simultaneously, can be increased by means of the method according to the invention. When the spectral signatures of the individual dyes overlap extensively, the wavelength range must be limited, according to the prior art, for separate detection of the fluorescence signals of individual dyes. This reduces the sensitivity of detection, i.e., increases the noise of the detectors, because greater amplification must be used. This is avoided by the method according to the invention. Further, nonspecific fluorescence signals, autofluorescence and fluorescence of the measuring device can be separated.

The method makes it possible to define a plurality of emission bands—even emission bands with overlapping spectra—which are recorded simultaneously during the measurement. Due to the spectral resolution that is used, the spectral components of the detected light signal can be determined over a large wavelength range. This allows determination of the spectral signature of a specific dye. Mixtures of several dyes can be distinguished based on the spectral signatures.

The signatures of the control and of the substrate are also determined in every measurement process for every measurement. In this way, the specific proportion of the measured signal can be determined in the measured sample signals. In so doing, nonspecific contributions which lead to false positive or false negative results are separated.

### BRIEF DESCRIPTION OF THE DRAWINGS

In the drawings:

FIG. 1 is a block diagram of the invention showing component measurement value acquisition;

FIG. 2 is a part block and part representational diagram for fluorescence and luminescence measurement;

FIG. 3 is a part block and part representational diagram for absorption measurements;

FIG. 4 is a block diagram embodiment form of the optical beam path of the unit shown in FIG. 1;

FIGS. 5 and 6 are block diagrams showing the evaluating arrangement of the detection arrangement;

FIG. 7 shows an example of a 96-well MTP (coordinate system) in representational form;

FIG. 8 illustrates a measuring and calculating flow chart in accordance with the invention;

FIG. 9 illustrates, in schematic form, unmixing based on two components in accordance with the invention;

FIG. 10 illustrates the setting, in graphical form, of the setting of the spectral range of interest;

FIG. 11 illustrates, in graphical form, the concept of ratio imaging; and

FIG. 12 illustrates, in representational form, fluorescence resonance energy transfer (FRET), in particular, the emission spectrum for FRET partners.

### 2. DESCRIPTION OF THE PREFERRED METHOD OF THE INVENTION

2.1 Method for Screening Active Ingredients

Underlying the method according to the invention is a spectrally split detection of the light emitted by the sample, wherein summing is carried out over different spectral components. The particular advantages of the invention are:
sensitive simultaneous detection of FL bands,
flexible configuration of the detection bands,
fast visualization of assay results,
the possibility of discriminating between contributions based on physiochemical characteristics of the compounds in the measured quantity.
   2.2 Description of the Implementation of the Method
   2.2.1 Definitions

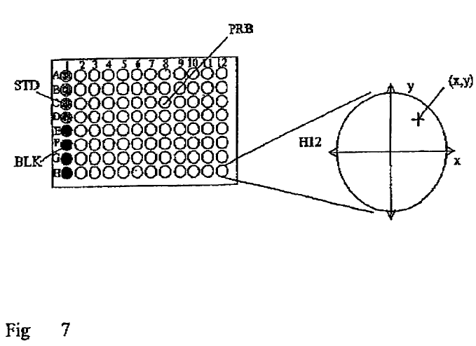

An MTP defines a coordinate system with different sample locations (coordinates A1, A2, . . . based on the SBS standard). FIG. 7 shows an example of a 96-well MTP. The drawing shows wells with coordinates 1-12 and A-H which can be approached individually by the scanning table quickly and with high accuracy by means of the X/Y positioning device. The coordinates are important for correlating with the measured samples. In the device according to the invention, the coordinates of the standardized MTP are pre-stored in the control unit of the scanning table. Accurate x/X positioning within a well, designated in this instance as H12 by way of example, is also possible.

Multiple measurements within a sample location yield sub-well data, as they are called, which are characterized by the same well coordinate but an additional spatial coordinate defining the position within a well. The analysis of sub-well data is important when there are inhomogeneities in the signal to be measured within a well which are detectable by means of the resolution of the system being used. These inhomogeneities make it possible to draw conclusions about sub-populations within a totality being investigated (e.g., cell cultures, groups of beads, or the like). The analysis of these sub-populations will not be discussed more fully hereinafter. However, this type of analysis is included in the method according to the invention.

Standard samples STD and blank samples (BLK), which contain neither STD nor substances to be investigated, are located on the MTP for determining the substrate signal. The samples to be investigated are located in the areas designated by PRB.

2.2.2 Determination of Spectral Regions (SRI—Spectral Range of Interest) From the Spectrum

In the method according to the invention, a spectrum of the control (STD) is recorded in a first step. The method for recording spectra and spectral regions is described in detail in the “Description of the Detection Unit”. The recorded spectrum is displayed graphically to the user at the control PC. After recording a spectrum in the manner described above, the user can select the spectral regions of interest (SRI) relevant for the investigation from the entire spectrum. This process is carried out once at the start of an experiment. The measurements are then carried out with the determined SRIs.

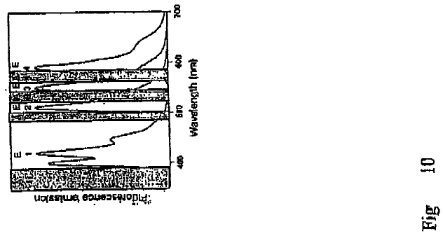

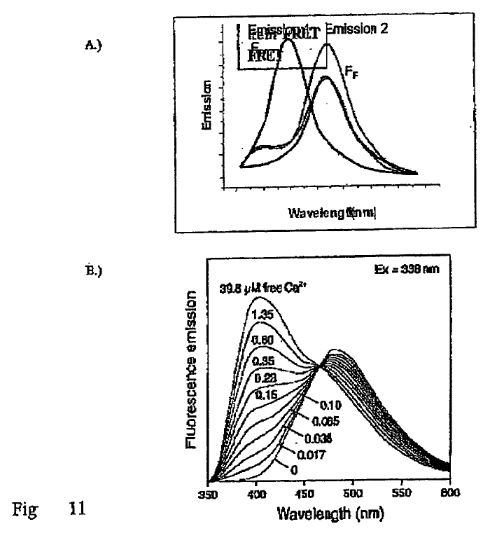

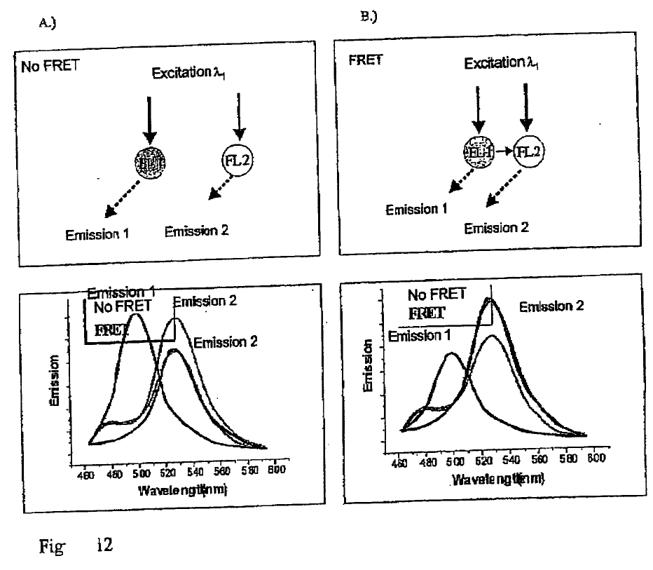

The setting of SRIs by the user can be carried out in the following manner, for example (FIG. 10). When more than one fluorescent dye is used, either a standard can contain all fluorescent dyes or there are a plurality of standards each of which contains a dye. In the latter case, the SRIs are determined individually for each standard. In the following example, four dyes are present in a STD well. After a spectral scan is made using all necessary excitation bands (A1-A4), summing channels can be formed between the individual excitation bands E1 to E4, according to FIG. 10, up to the maximum emission wavelength. These summing channels correspond to parts of the fluorescence bands of the individual dyes. In case of dyes with extensively overlapping emission bands, only a part of the spectral signal can be used in this way. Depending upon the choice of bandwidth for the summation of the emission light, this results in low sensitivity or insufficient specificity for a dye. A further development of the method according to the invention for discriminating between extensively overlapping fluorescences will be described later.

In addition to manual adjustment of the detection bands, automatic determination of the detection bands is also possible in that a spectrum of each individual FL component is measured as reference in an MTP well and the spectral components which lie above the comparator threshold and do not represent the excitation band of the light source are selected for detection. In a second method for setting the different SRIs, measurement of the fluorescence centroid is carried out. For this purpose, all individual channels that are irradiated by excitation light are switched off in the detector. Each SRI has a characteristic fluorescence centroid due to the altered emission characteristics of each of the dyes being used.

The different SRIs can accordingly differ through the position of the characteristic dye centroid and can be made visible separately.

Subsequently, an adjustment of the summing channels for the individual SRIs which is adapted specifically to the dye characteristics is carried out. In addition, any individual channels can also be switched off by the user. This is particularly useful for suppressing one or more excitation bands.

2.2.3 Calculation of the Measurement Values with Standards and Blanks

In the method according to the invention, the measured values are not determined and displayed as absolute values. Rather, the quantity to be determined is the intensity of a measured signal relative to a known reference sample and a blank sample. Standards (STD) and blanks (BLK) are determined by plate layout or use of calibration/reference plates between the assay plates. A standard/blank set is associated with every measurement value. Assay intensities are defined hereinafter as the measurement quantities which result from the calculation of measured intensities. An alternative method for determining the assay intensities which takes into account the spectral signature of the measured sample and reference is described in the following.

The SRIs mentioned above are determined beforehand based on the standard (STD). Definition: Assay intensities=(PRB-BLK)/(STD-BLK). STD, PRB, BLK can be individual measurements or averages from multiple measurements.

2.2.4 Simultaneous Measurement of a Plurality of SRIs

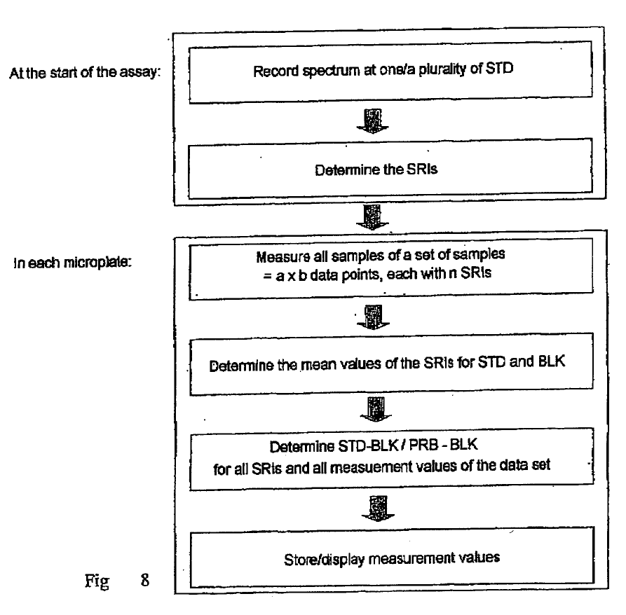

A measuring and calculating flowchart is shown in FIG. 8. The SRIs are determined at the start of the experiment. The method used for this purpose is described under “Determination of Spectral Regions”.

In the following, the measurement values are determined in the individual wells with reference to the SRIs in every microplate. Averages are determined from the measurement values of the BLK and STD wells (if more than one). The measurement values of the samples for the individual SRIs are calculated with the averages of STD and BLK. The data are displayed on and/or stored in the PC. Use of the method described herein with ratiometric methods, e.g., for FRET measurements (see above), consists in irradiation of the sample with light close to the excitation optimum of FRET partner 1 (donor excitation). The spectral region detected at the multichannel detector comprises the emission ranges of both FRET partners; by defining SRIs with the corresponding STD samples in the MTP (see above), the ratio of donor emission to acceptor emission can be measured simultaneously.

2.2.5 Unmixing

By using controls on the microplates, information about the spectral shape of the signal to be measured is available in every experiment.

Methods which make use of this information to suppress nonspecific components of the signal to be measured or to separate measurement values of overlapping spectra are described in the following.

Algorithms for analysis, e.g., for selective display of the contributors of individual dyes to the total fluorescence signal radiated from the sample, are described in the following. The analysis can be carried out quantitatively or qualitatively. In a quantitative analysis, the contribution (i.e., concentration) of every individual dye to the total fluorescence signal radiated from the sample is calculated for every well. Algorithms such as a linear unmixing analysis (Lansford, et al., Journal of Biomedical Optics 6(3), 311-318, (July 2001)) are used. The reference spectra which describe the fluorescence spectrum of an individual dye and are needed for the analysis are determined from the controls (STD, see FIG. 7) on the MTP.

In qualitative analysis, a classification is carried out, i.e., only the dye generating the greatest contribution to the total fluorescence signal radiated by the sample is associated with every well. Algorithms such as a principal component analysis (PCA, I. T. Joliffe, Principal Component Analysis, Springer-Verlag, New York, 1986) are used for this purpose. This type of algorithm allows the measurement values to be displayed on the control PC in the form of false colors—a masking of the image (dye mask) is obtained—and identical dyes are located in regions of the same color. This display allows co-localization of dyes at the same location within a well when analyzing (sub-)well data.

2.2.6 Unmixing with STD and BLK

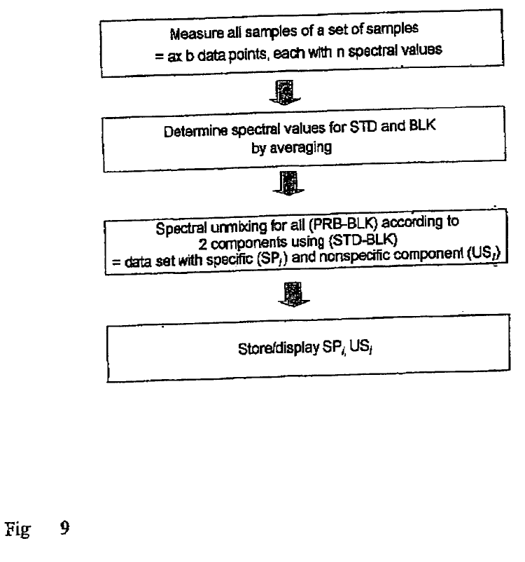

Unmixing-based on two components is shown in detail schematically in FIG. 9. The spectral values in the wells are determined for every microplate. In the STD and BLK wells, averaging is carried out in case of identical samples. Using STD, spectral unmixing according to two components is then carried out for every sample. This results in a data set containing the specific component SP (=STD component) and the nonspecific component US (=stray light, autofluorescence). These two data sets can then be displayed or stored.

Unmixing for Detection of WL Shifts

Another application of the unmixing method is with ratiometric methods when the emission spectrum of the dye changes depending upon existing ion concentrations or through spatial proximity to a bonding partner. Instead of determining the intensity in two narrow wavelength regions and forming the quotients, the ratio of the two components can be determined by unmixing. The advantage of the method consists in the fact that a substantially stronger signal is used for the measurement (because the entire spectral region is taken into account). This differs from “unmixing with STD and BLK” in that two STDs representing the extreme values of the spectrum (bonded vs. free, rich in ions vs. low in ions) are used as a reference. The unmixing then also supplies three components: two specific components (specific to the respective STDs) and the nonspecific component.

2.3 Description of the Preferred Embodiments of the Arrangement of the Invention

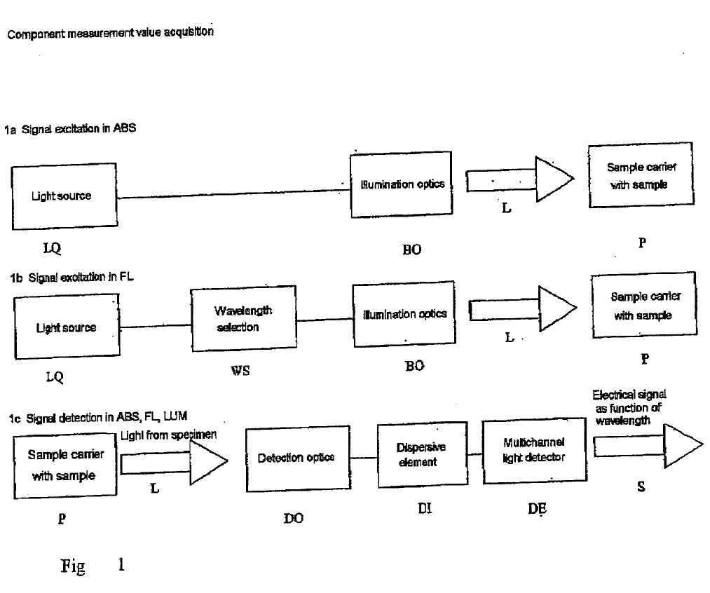

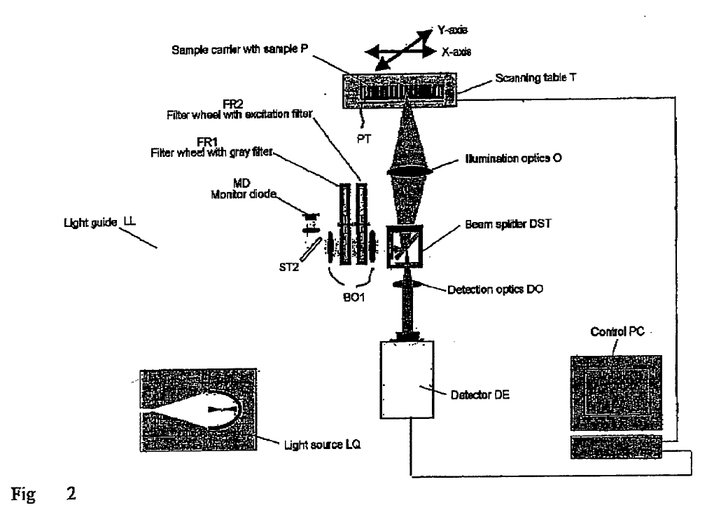

The most important elements of the invention are shown schematically in FIG. 1 in a block diagram.

For absorption measurements (ABS) (I a), the light L is focused in the sample by a broadband light source LQ and imaging optics BO and detected behind the sample.

With fluorescence measurements (FL), the specific excitation wavelength is selected by means of a wavelength-selective element WS from a broadband light source LQ and imaging optics BO are focused in the sample (1b).

The wavelength selection can be carried out, e.g., by suitable filters, prisms, dichroic splitter mirrors or combinations of these elements. Alternatively, monochromatic light sources are also possible. Examples include multiline lasers or combinations of individual lasers in a laser module. In this case, the wavelength selection is carried out, e.g., by means of AOTF or diffractive or dispersive elements.

In fluorescence detection (FIGS. 1b, c) and also in an analogous sense in FIGS. 1a and 1c for absorption, the emission light is split from the excitation light by means of an element for separating the excitation radiation from the detected radiation, e.g., a dichroic beam splitter. In transmitted light arrangements, e.g., for absorption measurements, and luminescence measurements (LUM), an element of this kind can also be dispensed with entirely. The light of the sample is imaged on a wavelength-dispersive element by means of imaging optics DO. The light is split into its spectral components by means of this dispersive element. Possible angular-dispersive elements include, e.g., prisms, gratings and acousto-optic elements. The light which is split into its spectral components by the dispersive element is subsequently imaged on a multichannel line detector DE. This line detector DE measures the emission signal depending upon the wavelength and converts it into electrical signals S. The individual channels are connected, i.e., summing is carried out over individual channels of the line detector, by means of a binning method according to the invention which will be described more fully in the following. In addition, a line filter can be arranged in front of the detection unit for suppressing the excitation wavelengths.

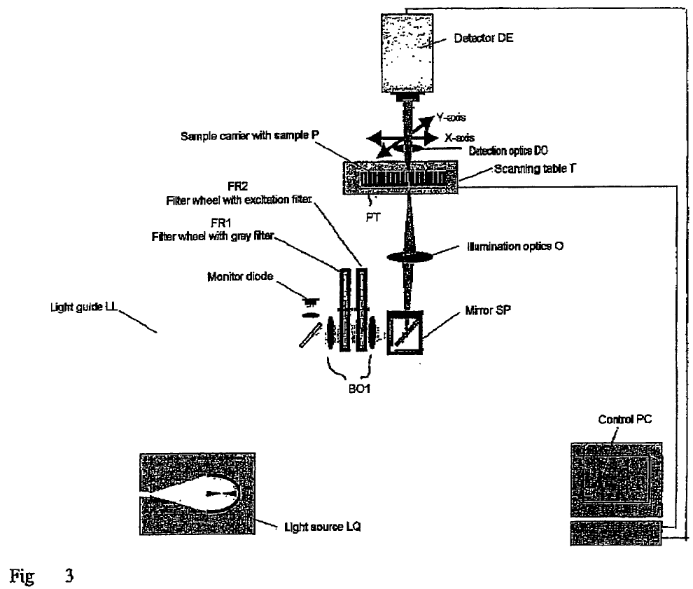

FIG. 2 shows an embodiment example of the entire arrangement. A sample carrier PT contains a sample P, in this case a microtiter plate, and is preferably constructed as a scanning table T, i.e., it executes a rapid movement at least in X/Y direction.

The MTP is illuminated from below through the vessel bottoms of the individual wells. The illumination comprises a light source LQ whose light is coupled into a light guide LL in this case and reaches the samples P via first illumination optics BO1 and filter wheels FR1, for intensity control by means of gray filters, and FR2, for wavelength selection by means of excitation filters and a dichroic beam splitter DST, and an objective O. The light coming from the respective samples reaches a detector DE through the beam splitter DST via detection optics DO.

A portion of the illumination light is coupled out by a beam splitter ST2 to a monitor diode MD for monitoring the laser output. A measurement can be carried out per sample (in the well of the MTP) or a plurality of measurements may be carried out in one well.

The samples P in the sample carrier PT are measured sequentially. For this purpose, the sample carrier PT with the sample is moved over the optical arrangement by a scanning table T. In an alternative arrangement, the sample would be stationary in a holder and the optical arrangement would move relative to the sample carrier by means of an xy scanner.

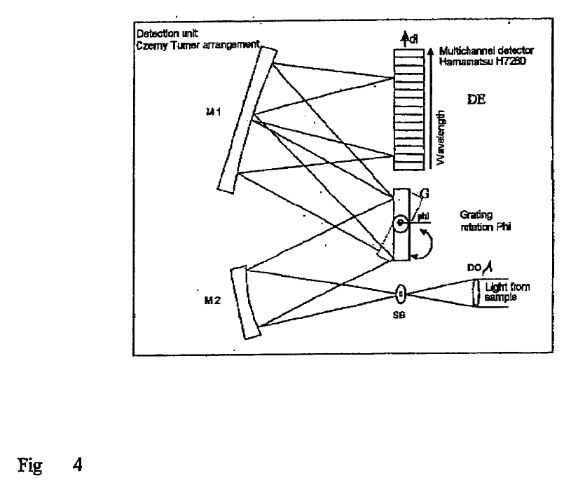

FIG. 2 shows an embodiment example for fluorescence and luminescence measurement. FIG. 3 shows an example for absorption measurements.

In this case, the detector arrangement DE is arranged over the sample. Instead of the beam splitter DST, a mirror SP is provided in this instance. The mirror SP couples in the illumination light laterally in the direction of the MTP.

At least the detector and the control of the scanning table are connected to a PC which can be connected to additional adjusting elements or evaluating elements.

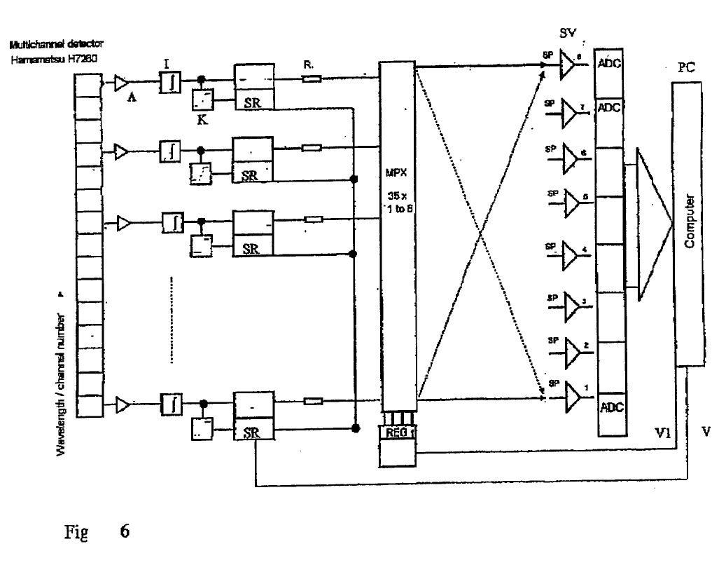

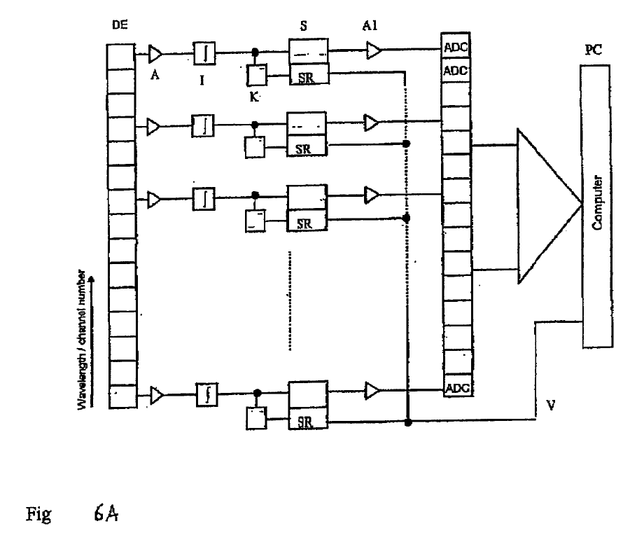

The detection arrangement DE can have an evaluating arrangement such as that shown in detail in the following particularly with reference to FIGS. 5 and 6.

2.3.1 Description of the Detection Unit

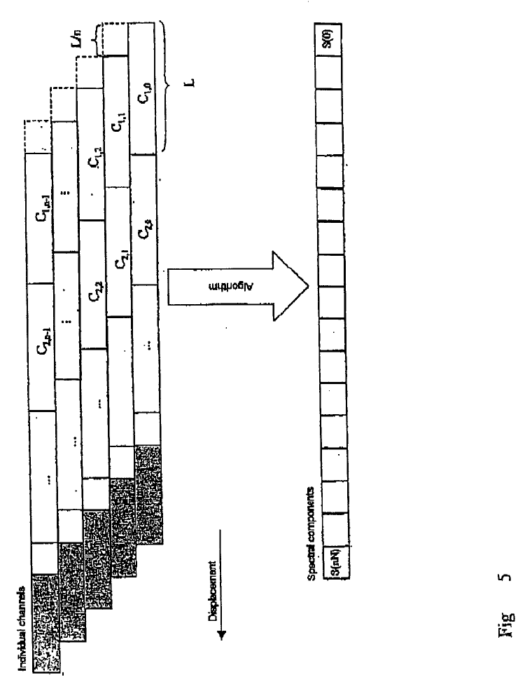

A possible embodiment form of the optical beam path of the detector unit shown in the block diagram in FIG. 1 is illustrated in FIG. 4. The construction is essentially a Czerny Turner construction. The light L of the sample is focused with the detection optics DO1 which receives the parallel light of the DO in FIGS. 2, 3. A baffle or stray-light diaphragm SB can be used, but is not absolutely necessary.

The first imaging mirror M2 collimates the fluorescent light. Subsequently, the light strikes a line grating G, for example, a grating with a line number of 651 lines per mm. The grating bends the light in different directions corresponding to its wavelength. The second imaging mirror M1 focuses the individual spectrally split wavelength components on the corresponding channels of the line detector DE. The use of a secondary electron multiplier array by Hamamatsu H7260 is especially advantageous. The detector has 32 channels and high sensitivity. The free spectral region of the embodiment form described above is approximately 350 nm. In this arrangement, the free spectral region is uniformly distributed to the 32 channels of the line detector resulting in an optical resolution of approximately 10 nm. Therefore, this arrangement is suitable for spectroscopy only conditionally. However, its use in a fast screening system is advantageous because the signal per detection channel is still relatively large due to the relatively broad spectral band that is detected. A shift of the free spectral region can be carried out in addition, for example, by rotating the grating by phi and/or by displacing the line receiver by dl in direction of the wavelength splitting (see FIG. 4).

In the embodiment form(s) described above, each individual channel advantageously detects a spectral band of the emission spectrum with a spectral width of approximately 10 nm. However, the emission of the dyes that is relevant for analysis extends over a wavelength range of several hundred nm. Therefore, in the arrangement according to the invention, the individual channels are summed corresponding to the fluorescence bands of the dyes that are used. For this purpose, a spectral scan, as it is called, is carried out in a first step on a reference sample located on the MTP to read out the information of the individual channels, e.g., as image information. When not all of the individual channels of the detector can be read out simultaneously, a sequential readout of the individual channels (multiplexing) is carried out according to the prior art.

For this purpose, the sample is advantageously irradiated with a plurality of excitation wavelengths simultaneously corresponding to the dyes that are used. The sum of the spectral components of the individual dyes found in the measured sample is recorded in this way.

Subsequently, the user can combine, that is, sum up, any of the individual channels to form detection bands (emission bands). The selection of the summation regions can be carried out, for example, by showing the signals of the sample in the individual channels in a histogram. The histogram represents the sum of all of the emission spectra of the dyes used in the sample. This summation is advantageously carried out corresponding to the emission spectra of the excited dyes, the respective excitation wavelengths are masked out and signals of different dyes in various detection bands are summed.

When eight channels can be read out simultaneously, for example, a summation is carried out over four channels in each instance using the 32 channel detector described above. The total N=32 channels are then read out in n=4 steps, the summation window being shifted in each instance by an individual channel (L/n=4/4=1). FIG. 5 shows schematically the different individual channels of the line detector, each in a line, to which N individual signals C correspond. Compared to a readout of the 32 individual channels in four steps, this procedure has the advantage that the signal information of all 32 pixels for the measurement time enters into the calculation of the individual spectral values. Otherwise, each detection channel is read out for only one fourth of the measurement time. It will be explained in the following how the individual spectral values can be obtained from the summing of the detector channels.

The measured signals of the individual channels are designated by cₖⱼ (shown as blocks in FIG. 5), where k=1 . . . N is the channel number and j=0. n−1 represents the multiples of the shift L/n. If the signal does not drop at the edge of the detector, the last individual channel of the detector can be covered (masked out), as is shown in gray in FIG. 5, in such a way that only a width of L/n is available for measurement. This is necessary for preventing artifacts during the calculation.

For calculating N times n spectral values Sₘ, sums of individual channels are subtracted according to the following algorithm:

$$\begin{matrix}\begin{matrix}\begin{matrix}\begin{matrix}\begin{matrix}\begin{matrix}\begin{matrix}\begin{matrix}\begin{matrix}\begin{matrix}\begin{matrix}\begin{matrix}\begin{matrix}\begin{matrix}\begin{matrix}\begin{matrix}\begin{matrix}{S}_{1} = {c}_{1 , {0}^{'}} = {\sum }_{i = 1}^{N} {c}_{i , 0} - {\sum }_{i = 1}^{N - 1} ci , 1 \\ {S}_{2} = {c}_{1 , {1}^{'}} = {\sum }_{i = 1}^{N} {c}_{i , 1} - {\sum }_{i = 1}^{N - 1} ci , 2\end{matrix} \\ ⋯\end{matrix} \\ {S}_{n - 1} = {c}_{1 , n - {2}^{'}} = {\sum }_{i = 1}^{N} {c}_{i , n - 2} - {\sum }_{i = 1}^{N - 1} {c}_{i , n - 1}\end{matrix} \\ {S}_{n} = {c}_{1 , n - {1}^{'}} = {\sum }_{i = 1}^{N} {c}_{i , n - 1} - {\sum }_{i = 1}^{N - 1} {c}_{i , 0} - {\sum }_{m = 1}^{n - 2} {c}_{N , m}\end{matrix} \\ ⋯\end{matrix} \\ {S}_{k \cdot n + 1} = {c}_{k , {0}^{'}} = {\sum }_{i = k}^{N} {c}_{i , 0} - {\sum }_{i = k}^{N - 1} {c}_{i , 1}\end{matrix} \\ {S}_{k \cdot n + 2} = {c}_{k , {1}^{'}} = {\sum }_{i = k}^{N} {c}_{i , 1} - {\sum }_{i = k}^{N - 1} {c}_{i , 2}\end{matrix} \\ ⋯\end{matrix} \\ {S}_{k \cdot n + j + 1} = {c}_{k , {j}^{'}} = {\sum }_{i = k}^{N} {c}_{i , j} - {\sum }_{i = k + 1}^{N - 1} {c}_{i , j + 1}\end{matrix} \\ ⋯\end{matrix} \\ {S}_{( k + 1 ) \cdot n + 1} = {c}_{k , n - {2}^{'}} = {\sum }_{i = k}^{N} {c}_{i , n - 2} - {\sum }_{i = k}^{N - 1} {c}_{i , n - 1}\end{matrix} \\ {S}_{( k + 1 ) \cdot n} = {c}_{k , n - {1}^{'}} = {\sum }_{i = k}^{N - 1} {c}_{i , n - 1} - {\sum }_{i = k + 1}^{N - 1} {c}_{i , 0} - {\sum }_{m = i}^{n - 2} {c}_{N , m}\end{matrix} \\ ⋯\end{matrix} \\ {S}_{N \cdot n - n} = {C}_{N , {0}^{′}} = {C}_{N , 0}\end{matrix} \\ {S}_{N \cdot n - n + 1} = {C}_{N , {1}^{′}} = {C}_{N , 1}\end{matrix} \\ ⋯\end{matrix} \\ {S}_{N \cdot n} = {C}_{N , n - {1}^{′}} = {C}_{N , n - 1}\end{matrix}$$

The spectral values S (intermediate values) calculated in this way can subsequently be represented graphically on the displayed image, e.g., during a spectral scan.

FIG. 6 schematically shows the summation over different individual channels and, therefore, the measurement of cₖⱼ. The signals of the individual channels are transformed into voltage signals by an amplifier A. The individual voltage signals are subsequently integrated in an integrator I during the pixel dwell time.

2.3.2 Simultaneous Recording of the Preselected Spectral Regions

The calculation of the emission bands can be carried out digitally or in analog. Both arrangements are described more fully in the following. An arrangement for digital calculation of the sum signal is shown schematically in FIG. 6A. In this case, the current at the anode of a multichannel PMT is converted to voltage and amplified through the first amplifier A (connected as current-voltage converter). The voltage is fed to an integrator I which integrates the signal over a corresponding time period (e.g., pixel dwell time).

For faster evaluation, the integrator I can be followed by a comparator K which, as a simple comparator, has a switching threshold such that a digital output signal is generated when this threshold is exceeded or which is constructed as a window comparator and then forms a digital output signal when the input signal lies between the upper and lower switching threshold or when the input signal lies outside (below or above) the switching thresholds. The comparator or window comparator can be arranged before or after the integrator. Circuit arrangements without an integrator (so-called amplifier mode) are also possible. With the amplifier mode arrangement, the comparator K is also present after corresponding level matching. The output of the comparator K serves as a control signal for a switch register SR which directly switches the active channels (on-line), or the state is conveyed to the computer via an additional connection V in order to make an individual selection of active channels (off-line). The output signal of the switch register SR is fed directly to another amplifier A1 for level matching for the subsequent analog-to-digital conversion AD. The A-D-converted values are transferred by suitable data transfer to a computer (PC or digital signal processor DSP) which carries out the calculation of the sum signal(s).

An equivalent of the arrangement in FIG. 6A based on analog data processing is shown in FIG. 6. In this case, the signals of the individual channels are again transformed into voltage signals by an amplifier A. The individual voltage signals are subsequently integrated in an integrator I during the pixel dwell time.

The integrator is followed by a comparator K which compares the integrated signal to a reference signal. In the event that the integrated signal was less than the comparator threshold, no fluorescence signal, or a fluorescence signal that is too small, would be measured in the corresponding individual channel. In such cases, the signal of the individual channel will not be further processed, since this channel only contributes a noise component to the total signal. In this case, the comparator actuates a switch via SR and the individual channel is switched off for the pixel that has just been measured. Accordingly, by means of the comparators in combination with the switches, the spectral region relevant for the image point that has just been measured is selected automatically.

The integrated voltage signal of the individual channels can subsequently be switched by a demultiplexer MPX connected with the switch register SR to different summing points by the register Reg1. FIG. 6 shows eight different summing points SP. The register Reg1 is controlled by the computer through a control line V1. Each summing point SP forms a part of the summing amplifier SV which carries out the summation of the selected individual channels. FIG. 6 shows a total of eight summing amplifiers SV. The sum signals are subsequently converted into digital signals by an analog-to-digital converter and are further processed by the computer or DSP. The summing amplifiers SV can also be operated with a variable nonlinear characteristic. In another arrangement (digital detection in FIG. 6A, analog detection in FIG. 6), the input signals of the individual detection channels are manipulated or distorted by a change in the gain of (A), by a change in the integration times of (I), by supplying an additional offset in front of the integrator and/or by means of digitally influencing the counted photons in a photon counting arrangement. Both methods can also be combined if desired.

A change in the summation pattern by V1 can be carried out imagewise after the recording or during the scanning of an image point/sample point or image row/image column. Requirements with regard to the switching speed of the MPX depend on the type of adjustment. For example, when adjustment is carried out by image point, the scan must be carried out within the integration period for this image point (that is, in several microseconds). When the adjustment is carried out by image, the scan must be carried out within several milliseconds to seconds. The calculation of the signals of the individual channels is carried out with the algorithm described above using cₖⱼ.

The arrangement according to the invention enables a fast change of the detection bands for multitracking applications, i.e., for changing the irradiation wavelength and/or intensity during measurement. The change can be carried out within a period of several μs. This also makes it possible to examine one and the same sample location with different detection bands, for example. Measurements requiring a fast change of the excitation light through polychromators or lasers with AOF can be realized by means of this fast change of detection bands.

The arrangement according to FIG. 6 has a number of advantages over the arrangement shown in FIG. 6A. The most striking advantage is that only the summing channels (that is, the detection bands of the dyes that are used) need be converted to digital data and sent to the computer. This minimizes the data rates to be processed by the computer. This is especially important when the method is applied in fast kinetics in the ms range in order to be able to record the dynamic processes taking place at extremely fast speeds. Further, when this method is used there are no limits on the quantity of individual channels of the line detector being used or, therefore, on the size of the detectable spectral region and/or the spectral resolution of the spectral sensor.

Further, the signal levels to be converted are substantially lower in the device shown in FIG. 6A. Therefore, the expected signal-to-noise ratio is lower.

An integrator circuit is preferably used in the two arrangements described above for detecting the individual channel signals. However, photon counting can also be carried out in the individual channels without restrictions and the photon counts can be added.

While the foregoing description and drawings represent the present invention, it will be obvious to those skilled in the art that various changes may be made therein without departing from the true spirit and scope of the present invention.

1-19. (cancelled).

20. A method for identifying fluorescing, luminescing and/or absorbing substances on and/or in sample carriers, particularly with high sample throughput in sample screening and/or in diagnostics, such as in the analysis of samples in microtiter plates comprising the steps of:
carrying out a spectral splitting of sample light;
carrying out detection in a plurality of detection channels; and
carrying out at least one summation and/or combination of signals of the individual channels for at least a portion of the detection channels.

21. The method according to claim 20, comprising arranging at least one standard sample and/or at least one blank sample on the sample carrier in addition to the substances to be examined.

22. The method according to claim 20, comprising recording a spectrum of at least one standard sample in a first step.

23. The method according to claim 20, comprising determining spectral regions of interest in which measurement is carried out automatically or by input means, based on measured standard spectra.

24. The method according to claim 20, comprising summing the detection channels of at least one spectral region of interest.

25. The method according to claim 20, comprising carrying out a change in the regions of summed detection channels and/or individual detection channels or switching off groups of channels.

26. The method according to claim 20, comprising determining a relative signal intensity of the substance is determined from the quotient (PRB-BLK)/(STD-BLK), where PRB is the measured signal of the substance, STD is the measured signal of the standard sample, BLK is the measured signal of the substrate (blank sample).

27. The method according to claim 20, comprising taking an average over a plurality of samples for STD and/or BLK.

28. The method according to claim 20, comprising carrying out a spectral unmixing according to at least two components for at least one substance based on standard samples.

29. The method according to claim 20, comprising taking the ratio of at least two components by unmixing.

30. The method according to claim 20, further comprising the step of providing a dispersive element, such as a grating or prism, and a receiver arrangement which is spatially resolving in at least one direction.

31. The method according to claim 30, wherein the receiver arrangement is a line detector.

32. The method according to claim 31, wherein the line detector is a multichannel PMT.

33. The method comprising carrying out a spectral weighting between a plurality of detection channels, a summation of the weighted channels of the signals of the detection channels.

34. The method according to claim 33, wherein the weighting curve is a straight line.

35. The method according to claim 33, wherein signals of detection channels are converted and digitally read out, and the weighting and summation are carried out digitally in a computing device.

36. The method according to claim 33, wherein the weighting and summation are carried out with analog data processing by means of a resistor cascade.

37. The method according to claim 36, wherein the resistors are adjustable.

38. The method and/or arrangement according to claim 33, wherein the weighting curve is adjustable.

39. An arrangement for identifying fluorescing, luminescing and/or absorbing substances on and/or in sample carriers, particularly with high sample throughput in sample screening and/or in diagnostics, such as in the analysis of samples in microtiter plates comprising:
means for carrying out a spectral splitting of sample light;
means for carrying out detection in a plurality of detection channels; and
means for carrying out at least one summation and/or carrying out a combination of signals of the individual channels for at least a portion of the detection channels.

40. The arrangement according to claim 39, wherein at least one standard sample (STD) and/or one blank sample (BLK) are/is arranged on the sample carrier in addition to the substances (PRB) to be examined.

41. The arrangement according to claim 39, wherein a spectrum of at least one standard sample is recorded in a first step.

42. The arrangement according to claim 39, wherein spectral regions of interest in which measurement is carried out automatically or by input means based on measured standard spectra.

43. The arrangement according to claim 39, wherein the detection channels of at least one spectral region of interest are summed.

44. The arrangement according to claim 39, wherein means are included for carrying out a change in the regions of summed detection channels and/or individual detection channels or for switching off groups of channels.

45. The arrangement according to claim 39, wherein means are included for determining a relative intensity of the substance from the quotient (PRB-BLK)/(STD-BLK), where PRB is the measured signal of the substance, STD is the measured signal of the standard sample, BLK is the measured signal of the substrate (blank sample).

46. The arrangement according to claim 45, including means for taking an average over a plurality of samples for STD and/or BLK.

47. The arrangement according to claim 39, including means for carrying out a spectral unmixing according to at least two components for at least one substance based on standard samples.

48. The arrangement according to claim 39, including taking the ratio of at least two components by unmixing.

49. The arrangement according to claim 39, wherein a dispersive element, such as a grating or prism, and a receiver arrangement which is spatially resolving in at least one direction are provided.

50. The arrangement according to claim 49, wherein the receiver arrangement is a line detector.

51. The arrangement according to claim 50, wherein the line detector is a multichannel PMT.

52. The arrangement according to claim 39, including means for carrying out a spectral weighting between a plurality of detection channels, a summation of the weighted channels of the signals of the detection channels and a summation of the detection channels.

53. The arrangement according to claim 52, wherein the weighting curve is a straight line.

54. The arrangement according to claim 52, including means for converting and digitally reading out signals of detection channels and the weighting and summation are carried out digitally in a computing device.

55. The arrangement according to claim 49, wherein the weighting and summation are carried out with analog data processing by a resistor cascade.

56. The arrangement according to claim 55, wherein the resistors are adjustable.

57. The arrangement according to claim 52, including means for adjusting the weighting curve.
